(19) United States
(12) Plant Patent Application Publication
(10) Pub. No.: US 2004/0205867 P1
(43) Pub. Date: Oct. 14, 2004

(54) Floribunda rose plant named 'Meilomit'

(75) Inventor: Alain A. Meilland, Antibes (FR)

Correspondence Address: BURNS DOANE SWECKER & MATHIS L L P, POST OFFICE BOX 1404, ALEXANDRIA, VA, 22313-1404, US

(73) Assignee: CP DELAWARE, INC., West Grove, PA (US)

(21) Appl. No.: 10/832,330

(22) Filed: Apr. 27, 2004

Related U.S. Application Data
(63) Continuation of application No. 10/299,914, filed on Nov. 20, 2002.
Application No. 10/299,914 is a continuation of application No. 09/573,163, filed on May 19, 2000, now abandoned.

(51) Int. Cl. A01H 5/00

(52) U.S. Cl. PLT/160

(57) ABSTRACT

A new and distinct variety of Floribunda rose plant is provided which forms in abundance attractive double blossoms that are Marigold Orange suffused with red in coloration. Such blossoms possess a light fragrance. The plant exhibits a bushy growth habit, and forms decorative dense dark green foliage that contrasts nicely with the blossom coloration. The plant also maintains well its attractive appearance even at the end of the blooming season. The new variety is particularly well suited for providing colorful ornamentation in the landscape.

This is a Continuation of U.S. Plant patent application Ser. No. 10/299,914, filed Nov. 20, 2002, which is a Continuation of U.S. Plant patent application Ser. No. 09/573,163, filed May 19, 2000 (now abandoned).

### BOTANICAL/COMMERCIAL CLASSIFICATION

Rosa hybrida/Floribunda Rose Plant

### VARIETAL DENOMINATION

cv. ‘Meilomit’

### SUMMARY OF THE INVENTION

The new Rosa hybrida variety of Floribunda rose plant was created by artificial pollination wherein two parents were crossed which previously had been studied in the hope that they would contribute the desired characteristics. The female parent (i.e., the seed parent) of the new variety was the ‘Meigurami’ variety (U.S. Plant Pat. No. 4,625). The male parent (i.e., the pollen parent) was the product of the cross of the ‘Allgold’ variety (U.S. Plant Pat. No. 1,665) and the ‘Yellow Cushion’ variety (U.S. Plant Pat. No., 2,849). The parentage of the new variety can be summarized as follows:

‘Meigurami’×(‘Allgold’×‘Yellow Cushion’).

The seeds resulting from the above pollination were sown and small plants were obtained which were physically and biologically different from each other. Selective study resulted in the identification of a single plant of the new variety.

It was found that the new variety of Floribunda rose plant of the present invention possesses the following combination of characteristics:

(a) forms in abundance attractive double blossoms that are Marigold Red suffused with red in coloration,

(b) exhibits a bushy growth habit,

(c) forms decorative dense dark green glossy foliage, and

(d) is particularly well suited for growing as ornamentation in parks and gardens.

The blossom coloration contrasts nicely with the dark green foliage. Also, the plant maintains well its attractive appearance even at the end of the blooming season.

The new variety of the present invention can be readily distinguished from its ancestors. For instance, the blossoms of the ‘Meigurami’ variety are smaller and are pink in coloration instead of Marigold Red suffused with red. The blossoms of the ‘Allgold’ variety are yellow. The ‘Yellow Cushion’ variety also forms yellow blossoms and displays more pointed buds.

The new variety well meets the needs of the horticultural industry and is particularly well suited for growing in the landscape.

The new variety has been found to undergo asexual propagation in France by a number of routes, including budding, grafting, and cuttage. Asexual propagation by the above-mentioned techniques in the South of France has shown that the characteristics of the new variety are stable and are strictly transmissible by such asexual propagation from one generation to another.

The new variety has been named the ‘Meilomit’ variety.

### BRIEF DESCRIPTION OF THE PHOTOGRAPH

The accompanying photograph shows as nearly true as it is reasonably possible to make the same, in a color illustration of this character, typical specimens of the plant parts of the new variety. The rose plants of the new variety were two years of age and were observed during September while budded on Rosa froebelli understock and growing outdoors at Le Cannet des Maures, Var, France. Dimensions in centimeters are indicated at the bottom of the photograph.

FIG. 1—illustrates a specimen of a young shoot;

FIG. 2—illustrates a specimen of a floral bud before the opening of the sepals;

FIG. 3—illustrates a specimen of a floral bud at the opening of the sepals;

FIG. 4—illustrates a specimen of a floral bud at the opening of the petals;

FIG. 5—illustrates a specimen of a flower in the course of opening;

FIG. 6—illustrates a specimen of an open flower—plan view—obverse;

FIG. 7—illustrates a specimen of an open flower—plan view—reverse;

FIG. 8—illustrates a specimen of a fully open flower—plan view—obverse;

FIG. 9—illustrates a specimen of a fully open flower—plan view—reverse;

FIG. 10—illustrates a specimen of a floral receptacle showing the arrangement of the stamens and pistils;

FIG. 11—illustrates a specimen of a floral receptacle showing the arrangement of the pistils (stamens removed);

FIG. 12—illustrates a specimen of a flowering stem;

FIG. 13—illustrates a specimen of a main branch;

FIG. 14—illustrates a specimen of a leaf with three leaflets—plan view—upper surface;

FIG. 15—illustrates a specimen of a leaf with five leaflets—plan view—under surface; and

FIG. 16—illustrates a leaf with seven leaflets—plan view—upper surface.

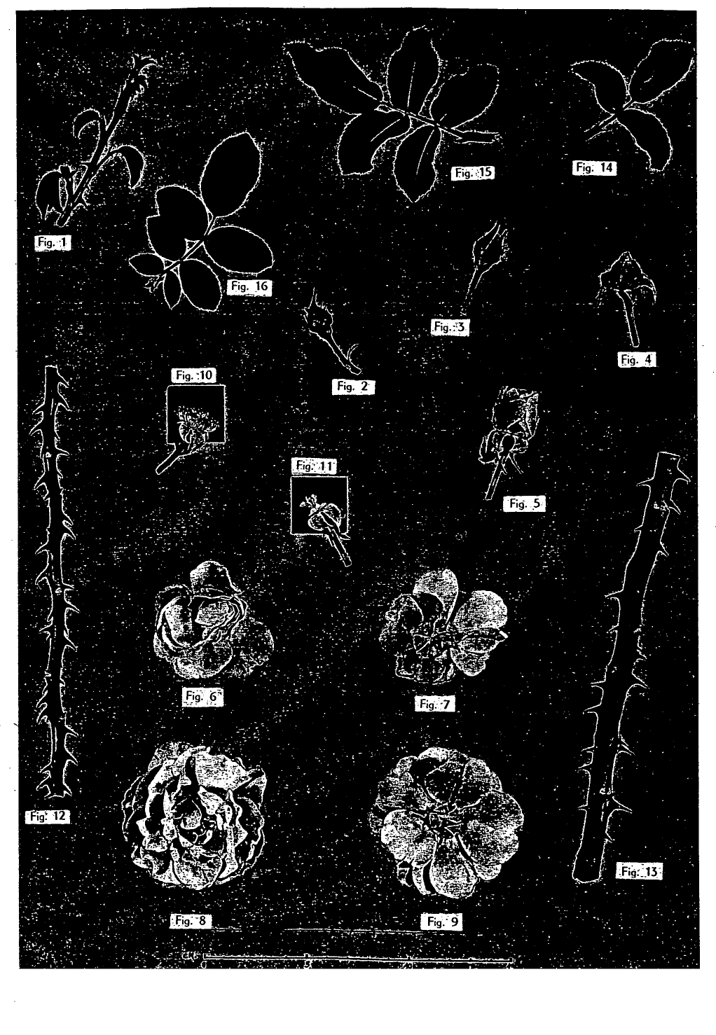

### DETAILED DESCRIPTION

The chart used in the identification of the colors is that of The Royal Horticultural Society (R.H.S. Colour Chart). The description is based on the observation of two year-old plants during October while budded on Rosa froebelli understock and growing outdoors at Le Cannet des Maures, Var, France. The coloration in common terms precedes reference to the chart in some instances.

Class: Floribunda.

Plant:

Height.—approximately 60 to 70 cm. on average at the end of the growing season.

Width.—approximately 70 to 80 cm. on average at the end of the growing season.

Habit.—bushy.

Branches:

Color.—young stems: Yellow-Green Group 147B with slight anthocyanin coloration near Greyed-Orange Group 176A. — adult wood: Green Group 138A.

Thorns.—size: medium (as illustrated). Mature thorns commonly are approximately 8 to 10 mm. in length on average, and immature thorns commonly measure approximately 6 to 8 mm. in length on average. — quantity: numerous (as illustrated). — color: mature thorns are near Greyed-Orange Group 166B and immature thorns are near Greyed-Orange Group 176A. — configuration: deeply concave.

LEAVES:

Stipules.—adnate, pectinate, approximately 1.6 cm. in length on average, approximately 0.4 cm in width, and the coloration is near Green Group 137C on the upper surface and near Green Group 137D on the under surface.

Petioles.—upper surface: glabrous and near Green Group 137A in coloration with slight anthocyanin coloration near Greyed-Orange Group 176A. — under surface: slightly glandular, and near Yellow-Green Group 148A in coloration.

Leaflets.—number: 3, 5, and 7. — shape: elliptic with an obtuse base and a tip that most commonly is obtuse and sometimes is cuspidate. — size: terminal leaflets commonly are approximately 4 cm. in length on average and approximately 2 to 2.5 cm. in width on average. — serration: minute and regular (as illustrated). — texture: leathery, smooth and glossy. — general appearance: very dense, dark green, and glossy. — color (young foliage): upper surface: dark green, near Green Group 136A. under surface: dark green, near Green Group 137A with some reddish anthocyanin coloration. — color (adult foliage): upper surface: dark green, near Green Group 139A. under surface: dark green, near Green Group 137A.

Inflorescence:

Number of flowers.—usually two to five flowers per stem.

Peduncle.—rigid and short, commonly with a few prickles, and with anthocyanin coloration near Greyed-Orange Group 176A.

Sepals.—upper surface: tomentose, commonly approximately 1.8 cm. in length and approximately 0.8 cm. in width on average, near Yellow-Green Group 148C and more or less suffused with reddish anthocyanin coloration. — under surface: glabrous, near Yellow-Green Group 146A and more or less suffused with reddish anthocyanin coloration. Some small extensions are present (as illustrated).

Buds.—shape: conical. — size: medium. — length: approximately 2 cm. on average.

Flower.—shape: flat cup-shaped. — diameter: commonly approximately 8.5 to 9.5 cm. — color (when opening begins): upper surface: Marigold Orange, near Orange Group 28C and lightly suffused with Red Group 52D. under surface: Marigold Orange, near Orange Group 28C and lightly suffused with Red Group 52D. — color (when blooming): upper surface: Marigold Orange, near Orange Group 28C and lightly suffused with Red Group 52D. under surface: Marigold Orange, near Orange Group 28C and lightly suffused with Red Group 52D. — color (at end of opening): upper surface: near Orange Group 29D at the base and near Red Group 49D at the tip. under surface: near Orange Group 29D at the base and near Red Group 55D at the tip. — petal basal spot: small and near Yellow Group 12B in coloration on both surfaces. — petaloids: none observed to date. — fragrance: light. — lasting quality: the blossoms commonly last approximately 10 days on the plant. — petal shape: rounded, commonly approximately 3 cm. in length on average and approximately 3.6 cm. in width on average, and commonly possess slight margin undulation. — petal number: approximately 25 to 27 on average. — stamen number: approximately 95 on average. — anthers: near Orange Group 29C in coloration. — filaments: near Yellow Group 8B in coloration and approximately 0.8 cm. in length on average. — pistils: approximately 32 on average. — stigmas: near Yellow Group 11A in coloration. — styles: near Yellow Group 11D in coloration and approximately 0.6 cm. in length on average. — receptacle: smooth, near Yellow-Green Group 146A in coloration, commonly approximately 5.5 to 6 cm. in length and width on average, and in longitudinal section in the shape of a pitcher. — hips: smooth, substantially round, commonly measure approximately 2 cm. on average, and are orange in coloration. — seeds: each hip commonly contains approximately 24 seeds on average that are cream-yellow in coloration.

Development:

Vegetation.—strong.

Blooming.—very abundant.

Resistance to diseases.—excellent with respect to Black Spot.

Aptitude to bear fruit.—good.

I claim:

1. A new and distinct variety of Floribunda rose plant characterized by the following combination of characteristics:
(a) forms in abundance attractive double blossoms that are Marigold Orange suffused with red in coloration,
(b) exhibits a bushy growth habit,
(c) forms decorative dense dark green glossy foliage, and
(d) is particularly well suited for growing as ornamentation in parks and gardens;
substantially as herein shown and described.
